(19) United States
(12) Patent Application Publication
(10) Pub. No.: US 2024/0189254 A1
(43) Pub. Date: Jun. 13, 2024

(54) Xylitol Topical Ocular Solution

(71) Applicant: GSX Pharma LLC, Newtown Square, PA (US)

(72) Inventors: Stephen Sinclair, Salt Lake City, UT (US); Joseph Greenberg, Newtown Square, PA (US)

(21) Appl. No.: 18/553,563

(22) PCT Filed: Jun. 14, 2022
(86) PCT No.: PCT/US2022/033445

§ 371 Date: Oct. 1, 2023

(51) Int. Cl. A61K 31/047; A61K 9/00; A61K 9/08; A61K 47/10; A61K 47/38; A61P 27/02

(52) U.S. Cl. CPC A61K 31/047; A61K 9/0048; A61K 9/08; A61K 47/10; A61K 47/38; A61P 27/02

(57) ABSTRACT

A solution for ocular application containing xylitol. Topically applied to the eye, the solution improves the quality of the ocular tear film, and reduces the incidence of infectious organisms, including bacterial abnormal colonization, acanthamoeba and an assortment of infective virus types including SARS, SARS-Cov-2. The same solution may also be applied to eyes of contact lens wearers and within the contact lens storage solutions to reduce the incidence of infection and in eyes with sicca syndromes demonstrating tear film poor lipid quantity or quality with meibomian gland dysfunction and in eyes demonstrating evidence of inflammation of the margins or posterior lid mucosal surfaces indicating blepharitis. The solution may be also applied to the lid margins with heat and ocular scrubbing massage in the case of eyes demonstrating blepharitis.

### PRIORITY CLAIM

This application claims priority from U.S. Provisional Application Ser. No. 63/210,566 filed Jun. 15, 2021.

### BACKGROUND OF THE INVENTION

The surface of the eye consists of a tissue layer of mucosa, termed the conjunctiva that wraps the underside of the lids and, with folding in the fornices, covers the eye to the limbal margins where it continues as a clear epithelium over the cornea. The cornea and conjunctiva are kept constantly moist by a tear film that provides optical clarity, lubrication, and a protective barrier to the cornea and conjunctiva against pathogenic and noxious agents. This film is composed of three layers each secreted by different glands: The surface lipids are contributed by meibomian glands in the tarsal plates of the upper and lower lids with orifices at the margins just inside of the lashes. The water portion is produced by tear glands under the lateral upper lid with orifices in the superior temporal fornix. The tear film provides nourishment to the surface epithelium of the conjunctiva and cornea maintaining clarity of the corneal epithelium and a uniform, smooth surface, optimal for ocular refraction with minimal diffraction. With each blink the tears are forced toward and into the naso-lacrimal ducts, with puncta in the nasal upper and lower lids that drain into the lacrimal sac beneath the nasal bone and (with valves that prevent backflow) into the nose (all contain mucosal linings with surface biofilm). Tear proteins within the tear film contribute to the anti-microbial and anti-inflammatory defense of the exposed ocular surface.

The tear film is spread and maintained by a reduction in surface tension caused by a lipid layer on the surface of the water fluid (with polar lipids that bind the lipid to the water surface and to a polarized Mucin layer that adheres the tear film to the epithelial surface of the eye and back surface of the lids. The tear film under normal visual scanning is swept over the surface of the conjunctiva and cornea by repeated blinking at rates approximating 15-17 per minute, but which may be reduced during periods of intensive visual concentration and fixation, such as while reading or performing visual tasks on a monitor (4.5/min) [Bentivoligio, 1997]. Therefore, there is a relationship between blinking rate and the inverse of the tear break-up time that we will term the tear film stabilization time. If the inter-blink time is prolonged, then the tear film may become destabilized and break apart exposing portions of the cornea or conjunctival surfaces. The epithelium of either can then develop defects, either superficial, punctate, or larger areas of erosion, or maybe subject to pealing of edges of the epithelium with exposure of the underlying basement membrane, inducing sensory burning, stinging and dry rubbing.

At the 2007 Dry Eye Workshop, it was noted that the corneal and conjunctival epithelia are in continuity, through ductal epithelia, with the acinar epithelia of the main and accessory lacrimal glands and the meibomian glands, which themselves arise as specialized invaginations from the ocular surface. Pathologic abnormalities may occur of any of the components that may result in a deficient tear film, or shortened tear-film stabilization time on the ocular surface. Inflammation appears to occur within the lids that causes the meibomian glands to secrete lipids of a paste like consistency rather than the usual fluid nature (similar to olive oil in consistency) [Nelson, 2011]; the reduction in the normal constituents usually results in a poor stabilization of the tear film and increased tear drying with a resultant shortened tear stabilization time and a reduced amount of a tear film that is hyperosmolar. The number of Mucin producing glands (goblet cells) or their output may also be reduced due to inflammation within the conjunctival mucosa that results in a number of syndromes of reduced adherence of the tear film to the epithelium [Danjo, 1998]. The lacrimal gland, also affected by the inflammation, may produce less tear quantity or altered soluble mucins. Finally, inflammation within the lids, termed blepharitis, caused or aggravated by resident bacteria, may cause scarring of the tarsal plates such that they cannot uniformly wipe the tears over the surface, or the bacteria may alter the tear components, especially the lipids, producing a break down into soaps that irritate the surfaces.

Prescribed and worn contact lenses must ride (ski) upon the surface biofilm of tears, constantly moving with each blink and forcing fresh tears beneath. Although the contact lenses allow some passage of oxygen through them, the contact lens movement on the surface is required to push sufficient fresh tears over the surface of the underlying corneal and conjunctival epithelium to provide sufficient oxygen and nutrients. Otherwise the corneal epithelium may die resulting in erosive areas (e.g. as occurs with inadvertent overnight contact lens wear while sleeping), or may stimulate neovascularization to grow over and through the surface to supply the ischemic epithelium (as occurs with contact lenses that are fitted too tightly such that the movement and pumping of tears is less than desirable).

### Ocular Sicca and Inflammatory Lid Syndromes

It should be remembered that normally there exists a relationship between tear dynamics and blink rate in which tear break-up precedes a blink by no more than 1 to 2 seconds. The lipid layer becomes unstable if the inter-blink interval is more than the TBUT, resulting in increased evaporation from the exposed surface. Ocular sicca syndromes in general cause significant symptoms of scratching irritation along with blurring that progress over time and are aggravated under conditions of low humidity or by visual tasks requiring prolonged focused fixations that are associated with reduced ocular motion and blinking rates (such as reading, monitor or television viewing, steady state driving such as upon highways, etc). Analysis of blink rate patterns in normal subjects reveals that the blink rate varies according to behavioral tasks with mean blink rates (blinks/min) at rest measuring approximately 15-17 blinks per minute, whereas during conversations that involve visual interactions this may increase to 26 per minute while during reading or monitor viewing they are reduced to 4-5 and with intensive concentration can be as low as 1 per minute. Provided the inter blink period is not severely prolonged, then the ocular surface epithelia remain wet and protected, but if there is a significant reduction in tear volume or instability of the tear meniscus which shortens the TBUT, even under normal blinking rates the epithelia can be damaged, but this is certainly aggravated by tasks requiring concentrated fixation.

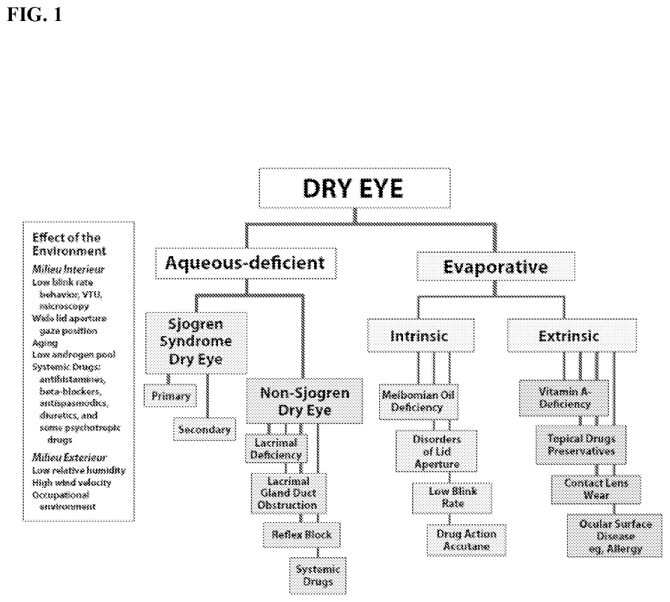

In general ocular sicca syndromes are classified into two categories, those manifesting a deficiency of the aqueous layer and those with TLL deficiencies (termed evaporative) as demonstrated in FIG. 1. (Classification of ocular sicca syndromes/disease states (after [Albertsmeyer, A. and I. Gipson, 2010: Surface System of the Eye]). However, it is now recognized that the effects are the result of complex inflammatory processes of all contributing glands that result in alterations of all components [Lu, 2022)]

In addition, abnormalities of the eyelids or the ocular surface itself can interfere with effective spreading of tears across with protection of the cornea and cause drying of the ocular surface; this phenomenon is often seen in ectropion or entropion of the tarsal plate, lid margin irregularity due to inflammation and scarring (blepharitis), exophthalmos due to thyroid disease, or corneal scarring. The conjunctival mucosa is a highly reactive tissue, which wraps over the surface of the eye and the posterior surface of the eyelids that is subjected to relative anerobic conditions within the fornixes compared with the aerobic conditions on the ocular surface, and hence variable micro-biofilm alterations within the various sectors of normal flora. The ocular surface and exposed gland ducts represent a highly reactive tissue, protected with a potent immune system, richly supplied by blood vessels and lymphatics and capable of significant inflammation with local humoral antibody secretion and T-lymphocyte cellular responses. The surface biofilm overlying the conjunctival and corneal surfaces predominantly harbor colonies of coryneabacter, propionbacter, non-caseating staph (such as staph albi, staph epidermidis), with minimal staph aureus, strep species and gram negatives, (predominantly those that do not produce toxins or acids). Several studies demonstrate that when the MAMs layer is abnormal or deficient (through reduced glycosyltated O-glycams) it is prone to injury with epithelial exposure and injury as clinically defined by Rose Bengal or fluorescein staining (Argueso, P., A. Tisdale, et al. 2006]). Bacteria may have both direct and indirect effects on the ocular surface and on meibomian gland function. These include direct effects on the production of toxic bacterial products (including lipases) and indirect effects on ocular surface homeostatic mechanisms, including matrix metalloproteinases (MMPs), macrophage function, and cytokine balance (Jacot J L, et al. 1985). The complexity and uncertainty of the role of bacteria in the MGD process, characterized by both infectious and inflammatory processes, has implications for appropriate recommendations for therapy. It may be that the excessive colonization of the lids, demonstrated in patients with blepharitis [Geerling, G, Tauber, J, et al, 2011], with coagulase-negative staphylococcus (Staphylococcus epidermidis), Staphylococcus aureus, Propionibacterium acnes or other microbes is an epiphenomenon, indicating the possibility that microbes find the altered eyelid environment in MGD more hospitable than that of the normal eyelid. Keratinization of the lid margin epithelium, the accumulation of keratinized cell debris, within and/or around the meibomian orifice, and the presence of abnormal lipids all provide a rich substrate for the resident bacterial microbiota. Thus, it is also possible that the subsequent release of toxic bacterial products such as lipases or the secondary production and release of proinflammatory cytokines is pathogenic. Excessive bacterial colonization may be pathogenic via preferential selection of certain microbial species. Although there certainly is disagreement as to whether direct and active bacterial infection is involved in the pathogenesis of MGD, bacterial products such as lipases and toxins (without infection) are still believed to be pathogenically relevant. Dougherty, J and McCulley, J, [1986] reported that the greatest bacterial lipolytic activity was found in those patients with meibomian gland abnormalities among the clinical groups of chronic blepharitis he examined.

The tear film in eyes manifesting the sicca syndromes is very often hypertonic. While the lacrimal gland secretes isotonic fluid, the osmolarity of the tear layer therefore varies and becomes hypertonic in normal conditions because of water evaporation if the lacrimal glands are not able to maintain an elevated rate of secretion sufficient to compensate for the water lost through evaporation; this is one of the sources of epithelial damage in dry eye disease, as the hyperosmolarity is one causative or aggravating factor of inflammation [Rolando, 2001]. The conjunctival surface disease in such sicca eyes is characterized by the loss of the mucus-producing goblet cells due to squamous metaplasia and hyper-keratinization of the conjunctiva, a common finding in advanced stages of the disease. Hyperosmotic tears also act as toxic agents toward the conjunctival epithelia, both by a direct osmotic mechanism and by mediating inflammation and very probably through alterations in the micro-biofilm with altered composition of the bacterial components.

### Effects of Xylitol on Non-Ocular Human Biofilm Bacterial Colonization

Bacteria in the oral cavity are transiently exposed to different sugars and live under constantly alternating “feast and famine” conditions. The rapid utilization of available sugars is possible through the phosphotransferase systems (PTS), which transports various sugars in bacterial cells with resultant phosphorylation [Tapiainen, T, 2010]. The PTS consists of enzyme components, a number of which are specific to the individual sugars. In addition to sugar metabolism, the PTS is a complex protein kinase system regulating metabolic processes and gene expression in many Gram-positive and Gram-negative bacteria, and its function is significant for oral streptococci such as Streptococcus mutans (S. mutans), which are dependent on sugars as the energy source.

Xylitol is a five-carbon polyol sugar alcohol, small amounts of which occur naturally in various fruits and berries. Xylitol disrupts the growth and virulence of foundation oral disease initiators, such as S. mutans, C. albicans, and P. gingivalis. As a prebiotic, it helps to establish, balance, and maintain a healthy microbiome, which supports innate immunity and disease resistance [Cannon, (2020)]; [Bahador A, et al, 2012].

Xylitol in several in vitro studies has been shown to significantly reduce the growth of multiple strains of bacteria [Badet C, et al, 2008]. The growth of S. mutans, a common oral bacterium was markedly reduced in spite of the presence of glucose or sucrose [Tapiainen, T. 2010]. Its effect on other oral bacteria such as Streptococcus salivarius and Streptococcus sanguis was modest by comparison, but still significant [Tapiainen, T. 2010]. Xylitol has also been shown to reduce the growth of Lactobacillus casei and some strains of Escherichia coli, Saccharomyces cerevisae and Salmonella typhii and to affect the sugar utilization of Haemophilus influenzae [Tapiainen, T. 2010].

Xylitol appears as well to prevent acute otitis media developing in children with sinusitis [Uhari, M., T. Tapiainen, et al, 2000]. The effect of xylitol on the growth of pathogenic bacteria in vitro has also been evaluated in an experiment using ten strains of S. pneumoniae and H. influenzae, five strains of M. catarrhalis and nine strains of beta-haemolytic streptococci [Tapiainen, T. 2010]. Xylitol induced a marked inhibition of pneumococcal growth, by 72% in the case of S. pneumoniae after exposure to a 5% xylitol solution and 39% in the presence of 1% xylitol, despite the availability of glucose in the growth medium. The extent of the effect on pneumococci was similar to that described in S. mutans exposed to xylitol [Tapiainen, T. 2010]. The growth of beta-haemolytic streptococci was more modestly impaired after exposure to 5% xylitol, whereas that of H. influenza and M. catarrhalis was minimally affected. In two clinical trials xylitol was found effective in preventing the development of acute otitis media with a daily dose of 8.4-10 g of xylitol given in five divided doses. The reduction in these 2-3 months follow-up trials was approximately 40% when chewing gum was used and approximately 30% with oral xylitol syrup.

The mechanism of action of xylitol on bacteria has been studied in detail, primarily for S. mutans, where the xylitol-induced growth reduction was inhibited in the presence of fructose but not in the presence of other sugars, suggesting that the effect is mediated through a fructose-dependent system [Tapiainen, T. 2010]. Subsequent research has demonstrated that xylitol is transported into the S. mutans bacteria and phosphorylated through a constitutive fructose PTS. Since this species is not able to utilize the xylitol phosphate as an energy source, the expulsion of the xylitol from the cell results in an energy-consuming futile xylitol cycle which, together with an intracellular accumulation of xylitol phosphate, results in inhibition of the growth of the evaluated S. mutans. Although most all studies have investigated the effects in S. mutans because of its oral and otopathology, since the PTS in bacteria regulates many metabolic processes and the expression of various genes, it is likely that in addition to growth retardation, xylitol also disturbs the metabolic processes in other similar bacteria. Certainly, if any such bacteria lacks the constitutive fructose PTS activity, the bacteria may become insensitive to xylitol.

Since bacteria adhere to host cells through carbohydrate-binding proteins, extracellular xylitol may not only affect growth but also may disturb the binding process of pathogens to epithelial surfaces by acting as a receptor analogue for the host cell, which could result in decreased adherence, another method that reduces potential colonization and infection. Xylitol has also been demonstrated to alter the polysaccharide synthesis in S. mutans, resulting in decreased bacterial adherence [Tapiainen, T. 2010]. In a 6% concentration Xylitol was capable of reducing the adherence of S. mutans, while a 5% concentration was sufficient to reduce the adherence of several of the main oto-pathogens, including S. pneumoniae and H. influenzae to epithelial cells [Tapiainen, T. 2010]. In another study, the effect of xylitol was compared to erythritol. For both polyols, the magnitude of the decrease in adherence observed was independent of the growth inhibition indicating that in the mouth, plaque accumulation of S. pneumonia occurs perhaps through a mechanism that is not dependent on growth inhibition [Söderling, E., T. Ekman, et al. 2008] [Soderling E, et al 2010]. In clinical trials, xylitol has been shown to decrease the occurrence of acute otitis media in day-care children. Exposure to xylitol lowered S. Pneumoniae supporting previous results where exposure to xylitol changed the ultrastructure of the pneumococcal capsule and could explain further the clinical efficacy of xylitol in preventing the otitis media [Kurola, P., T. Tapiainen, et al. (2009)]. In another study, the adherence of nine Streptococcus pneumoniae strains to epithelial cells was observed to be variable among strains, but there was excellent correlation between their adherent ability to artificial plates and binding to cells. Xylitol inhibited bacterial growth of all strains at concentrations ranging from 5% to 15%. At concentrations of 0.5 to 5% xylitol did not diminish significantly the adherence on epithelial cells. Therefore, at these lower concentrations the beneficial effect of xylitol in preventing some of the pneumococcal infections was thought to arise through growth inhibition rather than to an antiadhesive effect. [Ruiz V., 2011]

The ultrastructure of viable S. mutans bacteria appears also to be damaged after exposure to even small concentrations of xylitol [Tapiainen, T. 2010], with inhibition of protein synthesis, which implies that xylitol acts as a strong metabolic inhibitor for this species in the mouth. One strategy for treating dental caries is to suppress oral S. mutans (MS) with chlorhexidine, (CHX) mouth rinse. Oral MS levels, however, tend to quickly return to baseline values after a CHX rinse without further intervention. In one clinical study, after three months Xylitol in chewing gum appeared to prolong the effect of CHX therapy on oral MS and maintained long-term caries-pathogen suppression indicating that with currently available commercial products such as chewing gum, xylitol would result in significant caries inhibition [Hildebrandt, G. and B. Sparks (2000)].

Xylitol in concentrations at or exceeding the isotonic level of 4.5% acts also as an osmolyte. The hypertonic solution captures water on mucosal surfaces, which could be beneficial in conditions with abnormally high osmolarity, since a lowered salt concentration in the surface film is known to enhance the antimicrobial activity of the innate immunity system. In trials with frequent use of chewing gum, the concentrations of xylitol during usage were sufficiently high to have an antimicrobial effect, but the xylitol disappeared from the saliva within 15 minutes, suggesting that high peak concentrations even for shortened intervals may be more important for efficacy than the time for which the concentration exceeds the level needed for an antimicrobial effect. But trials evaluating this effect are still in progress.

### Effects of Xylitol on Other Bacterial Non-Ocular Mucosal Infections:

An invitro biofilm model of P. aeruginosa was subjected to treatment with xylitol in combination with lactoferrin. The combined treatment disrupted the structure of the P. aeruginosa biofilm and resulted in a greater than 2 log reduction in viability [Ammons, M., L. Ward, et al., 2008]. The findings indicated that a combined treatment of xylitol with lactoferrin significantly decreased the viability of established P. aeruginosa biofilms invitro and that the antimicrobial mechanism of this treatment included structural disruption with increased permeability of bacterial membranes.

### Effects of Xylitol on Ocular Mucosal Viral Inoculation and Infections Including HCoV

Although the coronaviruses (large, enveloped, single-stranded, positive-sense RNA viruses with a genome one of the largest found in any of the RNA viruses) have been recognized as human pathogens for about 50 years, specific antiviral drugs identified to prevent or treat HCoV infections have only recently been promulgated [World Health Organization, 2020]. This shortcoming became evident during the SARS-CoV outbreak in 2003 and was the start of numerous studies also following the subsequent SARS-Cov 2 outbreak of 2020 that included ocular modes of infection [Wu, et al, 2020]promising drug targets such as nonstructural proteins (eg, 3-chymotrypsin-like protease, papain-like protease, RNA-dependent RNA polymerase), which share homology with other novel coronaviruses (nCoVs) and drug targets that provide viral entry such as ACE2 [Ciaglia, E, et al, 2020] and the immune regulation pathways such as IL-6 [Sanders. 2020]. Xylitol, in concentrations expressed above, has been demonstrated to have significant antiviral effects for a number of mucosal infecting virions, including hRSV [Xu, 2016][Yin, S Y et al 2014], and SARS-CoV-2[Bansal, 2020], demonstrating in vitro significant, multiple log MAR reduction of virion concentrations within minutes of application and lasting for hours [Bansal, 2020]. Together with the MAM associated improvement in surface epithelia protection and with soluble kinases demonstrating ACE2 receptors (trapping the virus), xylitol would appear and has been demonstrated to reduce local epithelial infection and has been associated with reduced nasal and pulmonary secondary inflammation and compromise in animals with multiple types of viral mucosal and systemic inoculations [Canon, 2020] [Cheudieu, 2021]. Introduced into the tear film, it appears to have the same effects and would reduce inoculation and infection from rubbing an eye with contaminated fingers or from contaminated mist or aerosolized droplets derived from a nearby sneeze or cough[Seah, I, Agrawal, R, 2020].

### Effects of Xylitol on Infection by Acanthamoeba of Ocular Mucosal Infections

Acanthamoeba is a genus of free-living protozoa with a wide-spread distribution in the environment. Organisms of this genus are commonly found inhabiting soil and aquatic environments [Culbertson, C. G. 1971][Davies, P. G., D. A. Caron, and J. M. Sieburth. 1978][Kyle, D. E., and G. P. Noblet. 1986], but they have also been isolated from swimming pools [Mergeryan, H. 1991], tap water [Seal, D. V., F. Stapleton, and J. Dart. 1992] [Seal, D. V., C. M. Kirkness, 1999], bottled mineral water [Penland, R. L., and K. R. Wilhelmus, 1999], and even contact lens care solutions [Silvany, R. E., J. M. Dougherty]. Through these transfer mediums, the protozoa may cause severe corneal infections, with 95% of ocular acanthamoeba infection attributed to the contact lens solution [Silvany, R. E., J. M. Dougherty]. The organisms' life cycle is composed of two distinct stages: a motile, metabolically active trophozoite stage in which the organism is capable of multiplication and is sensitive to noxious stimuli, and a dormant cyst stage, in which the organism is resistant to desiccation, disinfection, and extremes of temperature.

Ocular infections due to Acanthamoeba were first reported in the early 1970s [Jones, D. B., G. S. Visvesvara, and N. M. Robinson. 1975] [Nagington, J., P. G. Watson, T. J. Playfair, J. McGill, B. R. Jones, and A. D. Steele. 1974], but it was not until the mid 1980s that a connection between contact lens wear and disease was established [Moore, M. B., J. P. McCulley, 1985]. Ledee and colleagues [Ledee, D. R., J. Hay, 1996] demonstrated a direct chain of causation of Acanthamoeba keratitis (AK) using DNA matching of isolates of Acanthamoeba griffini from the corneal scraping of an infected individual, the individual's lens storage case, and the individual's bathroom water supply. The use of ineffective lens disinfection systems [Stevenson, R., and D. V. Seal. 1998], homemade saline [Stehr-Green, J. K., T. M. Bailey, and G. S. Visvesvara. 1989], and tap water [Seal, D. V., F. Stapleton, and J. Dart. 1992] [Seal, D. V., C. M. Kirkness, 1999] and contamination of lens storage cases [Houang, E., D. Lam, D. Fan, and D. Seal. 2001][Larkin, D. F. P., S. Kilvington, and D. L. Easty. 1990] have all been cited as important risk factors for transmission of the disease.

The incidence of AK among contact lens wearers (CLWs) is not rare. Mathers and colleagues [Mathers, W. D., J. E. Sutphin, 1996] used tandem scanning confocal microscopy to screen the infected eyes of 217 patients with keratitis. The Acanthamoeba organism was suspected in 51 patients, and the presence was confirmed by cytology in 43. This led the investigators to conclude that the marked increase in acanthamoebic detection by tandem scanning confocal microscopy strongly suggested that the disease was more prevalent than initially suspected. A cohort study in Scotland quoted a peak incidence of AK in 1995 of 1 in 6,720 CLWs [Seal, D. V., C. M. Kirkness, 1999]. Similar studies in Holland in 1996 [Cheng, K. H., S. L. Leung, S. L, 1999] and Hong Kong during 1997 and 1998 [Lam, D., E. Houang, D. 2002] gave annual AK incidence rates of 1 in 200,000 CLWs and 1 in 33,000 CLWs, while Radford and colleagues [Radford, C. F., et al, 2002]carried out three multicenter questionnaire surveys on AK, reporting rates of approximately 1 in 32,260 [D. V. Seal, Stapleton, F, 1992].

The increased recognition of contact lens-associated AK during the early 1990s resulted in several investigations into the amoebicidal effects of contact lens solutions which have been thought to reduce the potential transmission rate. However, a major hurdle to be overcome when carrying out such a study is the enumeration of the amoeba. In a recent review of methods used to evaluate the effectiveness of contact lens solutions against Acanthamoeba, Buck and colleagues [Buck, S. L., R. A. Rosenthal, 1996] found that of the studies reviewed, 30% used no quantitative method and merely reported the presence or absence of viable amoebae. Several quantitative methods have been used, such as direct counting with a hemocytometer [Buck, S. L., R. A. Rosenthal, 1996, 1998][Connor, C. G., S. L. Hopkins, and R. D. Salisbury, 1991], standard plaque assay [Hugo, E. R., W. R. McLaughlin, 1991][Khunkitti, W., D. Lloyd, J. R, 1996], a quantitative microtiter method [Buck, S. L., R. A. Rosenthal, 1996, 2000], and enumeration of track-forming units developed on non-nutrient agar with a bacterial overlay [Kilvington, S. 1998]. With the exception of one study [Perrine, D., J. P. Chenu, P, 1995], a technique of organism enumeration that has been largely overlooked is the most probable number (MPN) technique, which is a means for estimating, without any direct counting, the density of organisms present within a liquid. Initially, mathematical equations for estimating the number of organisms present, based on the number of aliquots showing growth, were utilized, but more recently computers have now been used to develop more accurate MPN tables [Tillett, H. E., and R. Coleman. 1985][Tillett, H. E. 1987]. This MPN technique, which uses axenically produced trophozoites and mature, double-walled cysts, effectively has become the basis for a national standard of amoebicidal efficacy testing of multipurpose contact lens disinfecting solutions. Hydrogen peroxide solutions have been utilized because of their broad antimicrobial activity. However, the lenses must be neutralized before wear to avoid pronounced stinging and possible corneal damage. Neutralization is achieved by adding a catalyst during the disinfection process (one-step) or afterwards (two-step). In a recent study by Hughes and Kilvington, [Hughes, R, Kilvington, S. 2001] activities of commercial peroxide systems against both the trophozoites and cysts were compared. All disinfection systems were active against trophozoites. Although both two-step systems tested were cysticidal, the easier to use, one-step process, showed only some cysticidal activity. Only All-in-One proved effective against both trophozoites and cysts achieving the national standard recognized 3 log reduction for both trophozoites and cysts that is required by the ISO standard.

With the exception of Optifree Express, all CTLW solutions contain the preservative PHMB, with a concentration of 5 ppm in All-in-One and a concentration of 1 ppm in each of the remaining solutions. Several studies have investigated both the minimum trophozoite amoebicidal concentration (MTAC) and the minimum cysticidal concentration (MCC) of PHMB. Larkin et al. [Larkin, D. F. P., S. Kilvington, 1992], Hay et al. [Hay, J., C. M. Kirkness, D. V. Seal, and P. Wright. 1994] demonstrated the MTAC of PHMB to be 1 g/ml and the MCC to be 3 g/ml after 48 h of exposure with similar concentrations demonstrated by Elder et al. [Elder, M. J., S. Kilvington, 1994]. It was therefore not unexpected that All-in-One, which contains 5 g of PHMB per ml (5 ppm), was trophozoiticidal and cysticidal after 24 h of exposure; the solution also effectively killed both trophozoites and cysts within the MMRDT of 4 h. However, other solutions containing only 1 ppm PHMB would not have been expected and did not produce a cysticidal effect by 24 h.

In PMN testing of a number of Acanthamoeba species, Xylitol in concentrations ranging from 6% to 20% has appeared to potentiate the acanthamoeba cysticidal effects of PHMB. In order for a disinfectant like PHMB to be cysticidal, it must gain access to the trophozoite internalized within the cyst. The most obvious route is via the ostioles or the pores in the double cell wall of the cyst that connect the outer exocyst and inner endocyst, thus allowing the internalized amoeba to communicate with its outside environment. It is believed that the mucopolysaccharides of the ostioles are altered by xylitol that enhance the binding by PHMB to allow intracyst penetration of the disinfectant. The effectiveness of PHMB appears due to the binding of this highly positively charged molecule to the mucopolysaccharide, resulting in penetration and irreversible damage to the cell membrane and the cell contents. The cell damage caused by PHMB appears associated with leakage of calcium ions from the plasma membrane. Since the PHMB concentration used in many all-purpose solutions that were evaluated by Beattie [Beattie, Seal, 2003] (1 ppm [1 g/ml]) is at its minimum effective level (MTAC), the enhancing effects of Xylitol could be crucial and may improve cidal Acanthamoeba activities of the various commercial MPS's with the same concentration of PHMB (1 ppm) that have currently been utilized.

Effects of Xylitol on the Ocular Mucins of the Tear Film that Improve the Abnormalities of Adhesion, of Lid Mucosal Inflammation, and of Corneal Injury in the KCS Syndromes

As discussed above, multiple studies of specific mucins suggest alterations of both the amount and glycosylation of the membrane-associated mucins occur in many forms of dry eye (especially MUC5AC, MUC16 produced by goblet cells, that are lost in conjunctival epithelium in dry eye) [Albertsmeyer, A. and I. Gipson, 2010][Gipson, Hori, 2004][Gipson, 2004, 2007], which aggravates the friction injury with lid swiping on the underlying mucin complex and epithelia. Recent evidence has shown that such cell surface-associated mucins and their surface glycocalyx can contribute to the maintenance of the mucosal barrier integrity, preventing the penetrance of extracellular molecules and pathogens onto corneal and conjunctival surface epithelia and the progressive symptoms of DED. The role of MAM's in providing a protective cell surface barrier has also been shown in other wet-surfaced epithelia, such as those in the gastro-intestinal tract, [Mantelli and Argueso] also discussed above. Recent reports have described increased binding of Staph aureus to corneal limbal epithelial cell cultures lacking MUC16, supporting the role for cell surface-associated mucins in creating a physical barrier against the pathogens [Blalock and Spurr-Michaud, 2007, 2008]. Serous mucin producing cells, which generate MUC7, also secrete other bactericidal agents such as lysozyme. It has also been proposed that the small soluble mucins facilitate water retention and are “repulsive” or dis-adhesive and facilitate movement of cell proteins and debris through ducts, not only from the lacrimal gland acini through the long lacrimal duct to the tear film but also prevent colonization of ducts by pathogens. It appears that among the MAM's the glycosylation of MUC16, the expression of the gene itself, or the rate of shedding of the mucin from the cell surface is altered in multiple sicca syndromes [Guzman-Aranguez, et al, 2009]. Evidence suggesting that glycosylation of mucins is altered in cicatrizing ocular surface disease aggravating lid friction [Sumiyoshi, et al, 2008] has also been recognized as explained above. These results indicate that during the drying, keratinizing process of the ocular surface, the pattern of expression of glycosyltransferases that initiate O-glycosylation is altered, which leads to alteration in terminal carbohydrate structures on the mucins.

The mucin-rich environment of the intact corneal epithelium is thought to contribute to the prevention of Staphylococcus aureus infection. A study by Ricciuto, et al [Ricciuto, J., S. Heimer, et al. (2008)] examined whether O-glycans, which constitute the majority of the mucin surface glycocalyces, prevented bacterial adhesion and growth. Abrogation of mucin O-glycosylation in corneal epithelial cell cultures did not affect bacterial growth, but did indicate that mucin O-glycans contributed to the prevention of bacterial adherence to the apical surface of corneal epithelial cells, abrogating infection [Ricciuto, J., S. Heimer, et al. (2008)]especially in contact lens wearers [Ramamoorthy & Nichols, 2008]

### DETAILED DESCRIPTION OF THE INVENTION

The preferred embodiment of the present invention is the usage of a Xylitol solution in concentrations of 4.5% to 7% in ocular KCS for the purpose of re-establishing the membrane-associated mucin layer through improvement of the glycosylation of the O-glycan chains on the external tail of the epithelia attached mucins and also through the binding of lectin to conjunctival goblet cells. Alternatively, concentrations of from about 1% to about 8% may be employed. For contact lens solutions, up to about 10% xylitol may be employed. This has been shown to improve the altered MUC5AC glycosylation (as well as potentially other MAM's in which the ectodomains of MUC's 1,4, and 16 are reduced in their glycosylation in dry eyes [Albertsmeyer, A. and I. Gipson (2010)]. Glycosyltransferases are the enzymes responsible for the initiation and the elongation of the O-glycan chains on mucins as they transfer activated sugar residues to the proper acceptor. The composition and sequence of the carbohydrates in the O-glycan chain are influenced by the specific profile of glycosyltransferases expressed by the cell, their level of activity, and their position in the intracellular Golgi. The O-glycan chains are extended by addition of polylactosamine and/or by any of a large repertoire of terminal carbohydrates, which includes, intra- or extra-cellular glycosyl polyol sugars. O-Glycans on mucins occur as a micro-heterogeneous population of neutral, sialylated, and sulfated oligosaccharides. Little is known about the extracellular modification of the MAM glycocalyx, but it appears significantly, adversely altered and thinned in a number of sicca syndromes. [Gipson, I and Argueso, P. 2003][Argueso, P, Tisdale, 2006]

The addition to the O-glycan glycocalyx occurring in the presence of xylitol appears to occur extracellular also by non-enzymatic glycosylation or by upregulation of GalNAc-transferases that results in an increase in the density of O-glycans. This improved glycosylation has been shown to improve the barrier for a number of bacterial strains (reduced adherence) in non-ocular mucosa because of the improvement in the extended, rigid structure of the Mucin molecule or by charge repulsion, due to the abundance of negatively charged sialic acids. It is thought that microabrasions on the apical epithelial cell surface caused by contact lens shear stress or with dryness as with the multitude of sicca syndromes, or epithelial keratinization (such as occurs over filtering blebs, especially those following the use of mitomycin C) alter the mucin O-glycan composition with thinning of the epithelial glycocalyx, and contribute to bacterial adhesion. However non-enzymatic glycosylation, we recognize, will require maintaining a relative high level of xylitol in the mucin environment, (i.e., frequent drop application, which will be addressed below)

Xylitol is also believed to increase non-enzymatic glycosylation of certain sphingolipids or glycosphingolipids that are necessary with lipocalins to reduce surface tension and improve spreading of tears over the ocular surface [Bron, et al, 2004] or bind to a novel class of lipids recently identified in human meibum, very long chain (O-acyl)-hydroxy fatty acids that appear to act in the formation of an intermediate surfactant lipid sublayer between the thick outermost nonpolar lipid sublayer and the aqueous layer of the TF. By definition, therefore, such polar lipids are relatively water-soluble. They include short chain fatty acids, hydroxylated fatty acids, hydroxy-ceramides (OH-Cer), monoacyl glycerols (MAGs), glycosylated lipids, phospholipids, and others. These lipids tend to have relatively high hydrophilic-to-lipophilic balance (HLB), which is an objective physicochemical parameter used to describe partitioning of solubilized molecules between polar (aqueous) and nonpolar (oil) subphases. [Green-Church, K., Butovich, I, Willcox, M., et al. (2011)]

Although bacterial infection has not been considered as an important factor in obstructive “mybomian gland dysfunction” occurring in the vast majority of sicca syndromes (please see above), bacterial products such as lipases and toxins (without infection), and the induced inflammatory reactions are still believed to be pathogenically relevant [Nichols, et al, 2011]. Dougherty and McCulley reported that the greatest bacterial lipolytic activity was found in those patients with meibomian gland abnormality among six clinical groups of chronic blepharitis [Dougherty, J. and J. McCulley (1986)]. Bacterial lipases do alter the lipid composition, influencing the physical characteristics of the tear film and causing evaporative dry eye progressive symptoms [Knop, E., N. Knop, et al. (2011)].

Topical applications at 4% to 7% Xylitol concentrations with usage at 2-5 times per day in limited human studies have been demonstrated to reduce corneal SPK, improve symptoms and inflammation as recorded by imaging of the retro-palpebral and ocular surface mucosa as well as of the Meibomian gland obstruction. Similar to the nasopharyngx, it should demonstrate diminished pathogenic strep species, staph aureus, and some gram negatives including H Flu, as well as Pseudomonas, by the mechanisms observed in in the nasopharyngeal and otic mucosa and potentially improving safety of eyes at risk (trabeculectomy, bleb-filtered eyes and eyes undergoing injections), studies of which are underway along with improvement due to improved fluometry of tears on the ocular surface from improvement in both soluble and membrane-associated mucins.

### Aqueous Delivery and Adherence/Stabilization Promoter Vehicles

Because of the desire to maintain a constant concentration of xylitol in the tear film complex, as discussed above, in order to promote the extracellular non-enzymatic glycosylation of O-glycans or by upregulation of GalNAc-transferases that result in an increase in the density of O-glycans, as well as the demonstrated antiviral efficacy the xylitol mixture may include a vehicle to promote stabilization in the muco-adhesive matrix.

### DuraSite

The DuraSite vehicle (http://www.insitevision.com/durasite) appears to offer such a system and can be customized to deliver a wide variety of potential drug agents. DuraSite is a proprietary drug delivery vehicle that stabilizes small molecules in such a polymeric muco-adhesive matrix. The topical ophthalmic solution can be described as a gel forming drop, which extends the residence time of the drug relative to conventional eye drops.

The addition of the DuraSite drug delivery vehicle to azithromycin has been demonstrated to increase the drug's contact time with the ocular surface for several hours. This permits products like AzaSite to achieve high, prolonged tear-film and conjunctival concentrations of the antibiotic, minimizing dosing frequency and potentially reducing adverse side effects. The Company is in process of investigating the addition of DuraSite delivery for a Xylitol ophthalmic complex.

### Anden Anchor Enzyme Complex

An alternative embodiment of the present invention is the Anden Anchor-Enzyme Complex (AEC™) that has demonstrated efficacy in the mouth to reduce bacterial induced plaque formation. The AEC™ platform does not appear to encourage resistance or kill beneficial bacteria. At the core of the AEC™ platform is a protein with both an enzymatic function (catalytic domain) and retention function (binding domain) within a single molecule.

The increased retention time in the oral cavity ensures long lasting action of various treatments to inhibit the formation of new plaque with the dual capability of limiting bacterial colony size and proliferation while maintaining the natural balance or ratio among species. Plans and protocols are in progress to investigate a development program for ocular application. The results from the companion animal product development program will also serve as the basis of the preclinical development program for a human geriatric product as discussed above.

### Iota Carrageenan

Carrageenans, linear sulfated polysaccharides that are often extracted from red seaweed, have been used extensively for years in the cosmetic and pharmaceutical industry as suspension and emulsion stabilizers. Their antiviral capacity has been described decades ago and has been experimentally confirmed on herpes virus type 1 and 2, human papiloma virus, H1N1 influenza virus, dengue virus, rhinovirus, hepatitis A virus, enteroviruses, and coronaviruses [Bansal, (2020)]. Iota-carrageenan inhibits several viruses based on its interaction with the surface of viral particles, thus preventing them from entering cells and trapping the viral particles released from the infected cells [Bansal, 2020]. Iota-carrageenan, formulated into a nasal spray, has proved to be safe and effective against multiple viruses demonstrating invitro inhibition of rhinovirus, influenza, and common-cold as well as Sars-Cov-2 [Bansal, (2020)]. Both iota-carrageenan and xylitol combined appear to be safe for humans in nasal formulations already on the market for use in children and adults.

Inactive constituents in the preferred embodiment:

### Water

### Thickening Agents and Emulsifiers

Sodium Hyaluronate
CMC=carboxyl methylcellulose sodium
HPMC=hydroxypropyl methylcellulose, hypromellose
Polyethylene glycol

### Preservatives

Preservatives are optionally included. Superficial punctate keratitis, as noted above, may be observed to extend beneath the palpebral fissure producing conjunctival staining in the lower nasal part where irritant eyedrops tend to accumulate, and are possibly indicative of the toxic effects. Many toxic effects of topical treatments are not clinically evident, however, and may only be assessed by discrete signs, such as more rapid tear break-up time, or may remain subclinical, evidenced only on impression cytology specimens or conjunctival biopsies. Squamous metaplasia, inflammatory infiltrates, abnormal epithelial expression of immune mediators and antigens, and increased apoptotic markers have, therefore, been described in the conjunctiva of patients receiving prolonged topical treatments [Baudouin, C. (2001)]. Current preservatives in other artificial tear solutions include:
Polexitonium
GenAqua
Polyhexamethylene Biguanide
Sodium Perborate

### Buffers

Other common additives used in artificial tear preparations are buffers, which have the purpose of maintaining the pH of human natural tears (7.4) as closely as possible when they are applied to the ocular surface. This is important, as it has been widely demonstrated that the pH of the tear film should be kept constant to maintain the normal function of the epithelial cells on the ocular surface. It has also been shown that often the pH decreases after instillation of eyedrops, and then rapidly becomes more alkaline before normalizing after approximately 2 minutes. Since the chemical buffering capacity of natural tears depends mostly on bicarbonates, this and also other components (phosphates, acetates, citrates, borates, sodium hydroxide) are frequently added to artificial tears in an attempt to make them slightly alkaline, since the more alkaline solutions appear to be more comfortable than neutral or acidic preparations [Calonge, M. (2001)]

### Hypotonic Formulations to Reduce Tear Hyperosmolarity

Hypotonic electrolyte-based formulations have been developed based on the recognition of the importance of tear osmolarity and electrolytes in maintaining the ocular surface. As discussed above, tear film osmolarity and tear electrolyte (sodium, potassium, calcium, magnesium, bicarbonate) levels have been demonstrated to be increased in dry eye states caused by meibomian and/or lacrimal gland disease and malproduction. Bicarbonate, especially, appears to be an essential component in the recovery of the damaged corneal epithelial barrier and in the maintenance of normal ultrastructure. One artificial tear formulation that is not just hypotonic but also derives a unique electrolyte-based composition, seems to increase corneal glycogen and conjunctival goblet cell density in a rabbit model of KCS and to decrease rose bengal staining and tear film osmolarity in dry eye patients. [Calonge, M. (2001)]. This is being investigated in the company's current clinical formulations.

### Preferred Embodiment Formulation

| Component | Vol. Percent |
|---|---|
| Purified Water | 92.6475 |
| Xylitol | 4.50 |
| PEG-400 | 0.40 |
| Hydroxypropyl | 1.75 |
| methyl Cellulose | |
| Na Perborate | 0.0025 |
| NaCl | 0.70 |

The preferred embodiment formulation exhibits a viscosity of about 6 cps at standard temperature and pressure. Alternate embodiments may vary the amount of xylitol from about 1% to about 8%, with a preferred range of from about 4% to about 7%.

While the invention has been described in its preferred embodiments, it is to be understood that the words which have been used are words of description rather than of limitation and that changes may be made within the purview of the appended claims without departing from the true scope and spirit of the invention in its broader aspects. Rather, various modifications may he made in the details within the scope and range of equivalents of the claims and without departing from the spirit of the invention. The inventors further require that the scope accorded their claims be in accordance with the broadest possible construction available under the law as it exists on the date of filing hereof (and of the application from which this application obtains priority, if any) and that no narrowing of the scope of the appended claims be allowed due to subsequent changes in the law, as such a narrowing would constitute an ex post facto adjudication, and a taking without due process or just compensation.

We claim as our invention:

1. A substance in the form of an aqueous solution comprising:
(a) xylitol, in a volume concentration from about 1% to about 8%;
(b) an optional aqueous delivery and adherence/stabilization promoter;
(c) an optional thickener comprising one or more materials selected from the group comprising white petrolatum, mineral oil, sodium hyaluronate, carboxy methycellulose sodium, hydroxypropyl methylcellulose, hypromellose, and propylene glycol;
(d) an optional preservative;
(e) a buffer adapted to maintain the solution near to the pH of natural human tears;
(f) optional ionic species comprising one or more ions selected from the group of sodium, potassium, calcium, magnesium and bicarbonate, in an amount sufficient-necessary to render the aqueous solution isotonic with human tears; and
(f) optionally, vitamin A;
for the treatment of diseases characterized by defects in the ocular tear film.

2. An aqueous solution for extra-ocular storage of contact lenses that demonstrates reduction in the incidence of bacterial, viral or acanthamoeba growth on the surface of the contact lenses comprising:
(a) xylitol, in a volume concentration of from about 6% to about 10%;
(b) an optional preservative
(d) a buffer to maintain the solution near to the pH of natural human tears; and
(e) optional ionic species comprising one or more ions selected from the group of sodium, potassium, calcium, magnesium and bicarbonate, in an amount necessary to render the aqueous solution hypotonic.

3. A substance in the form of an aqueous solution comprising:
(a) xylitol, in a volume concentration of from about 4.5% to about 8%;
(b) an optional aqueous delivery and adherence/stabilization promotion vehicle;
(c) an optional thickener or emulsifier comprising one or more materials selected from the group of white petrolatum, mineral oil, sodium hyaluronate, carboxy methycellulose sodium, hydroxypropyl methylcellulose, hypromellose, and propylene glycol;
(d) an optional preservative;
(e) a buffer to maintain the solution near to the pH of natural human tears; and
(f) optional ionic species comprising one or more ions selected from the group of sodium, potassium, calcium, magnesium and bicarbonate, in an amount necessary to render the aqueous solution isotonic with human tearshypotonic;
for the treatment of ocular diseases by applying to the eyelids as a scrub.

4. A substance in the form of an aqueous solution comprising:
(a) xylitol, in a volume concentration from about 1% to about 8%;
(b) an optional aqueous delivery and adherence/stabilization promoter;
(c) an optional thickener comprising one or more materials selected from the group comprising white petrolatum, mineral oil, sodium hyaluronate, carboxy methycellulose sodium, hydroxypropyl methylcellulose, hypromellose, and propylene glycol;
(d) an optional preservative;
(e) a buffer adapted to maintain the solution near to the pH of natural human tears;
(f) optional ionic species comprising one or more ions selected from the group of sodium, potassium, calcium, magnesium and bicarbonate, in an amount necessary to render the aqueous solution isotonic with human tears; and
(g) optionally, vitamin A;
for the prevention and treatment of viral diseases.
